(19) United States
(12) Patent Application Publication
(10) Pub. No.: US 2005/0015147 A1
(43) Pub. Date: Jan. 20, 2005

(54) Spinal implants

(76) Inventors: Jeffrey Schwardt, Morristown, NJ (US); Paul D'Antonio, Morristown, NJ (US)

Correspondence Address: KARAN SINGH, 729 SEVENTH AVENUE, 14TH FLOOR, NEW YORK, NY, 10019, US

(21) Appl. No.: 10/499,228

(22) PCT Filed: Dec. 18, 2002
(86) PCT No.: PCT/US02/40624

Related U.S. Application Data
(60) Provisional application No. 60/341,360, filed on Dec. 18, 2001.

(51) Int. Cl. A61F 2/44

(52) U.S. Cl. 623/17.11

(57) ABSTRACT

Spinal implants having at least one of several attributes, including a surface layer of demineralized bone, a beveled edge, and channels in the faces in contact with adjacent vertebral bodies. Preferably, the implant has a generally planar top surface, a generally planar bottom surface, and a side surface, wherein at least one of the top and bottom surfaces is demineralized to a depth of from 0.8 mm to 3 mm. Preferably, the top and bottom surfaces of the implant are textured. Also preferably, the implant is disk shaped and comprises an insertion side extending at least 10% of the circumference of the implant, wherein the edge formed by the insertion side and the top surface, and the edge formed by the insertion side and the bottom surface, are beveled.

### FIELD OF THE INVENTION

The present invention generally relates to devices for use in orthopedic surgical procedures, and more particularly to spinal implants and methods of manufacture.

### BACKGROUND OF THE INVENTION

During the course of treating many spinal disorders it frequently becomes necessary to permanently secure vertebrae in a relatively fixed position. Such disorders may arise as a result of disease, trauma, or congenital deformation. One group of such disorders results from the degeneration of one or more intervertebral disks, which are layers of fibrocartilage between the adjacent vertebrae. Degeneration may result in shrinkage or displacement (“slipping” or herniation) of the disk. As a result, the spinal cord and emergent nerves can become compressed, due to misalignment of the vertebrae or pressure from displaced disk material, with chronic and sometimes debilitating, neck, back, and peripheral pain.

One method of treatment for intervertebral disk degeneration involves surgical decompression of the affected vertebrae and nerves, and removal of the disk material (diskectomy). The adjoining vertebral bodies are then fused or otherwise fixed. In particular, in an anterior interbody fusion some or all of a disk is replaced with an implant by using an anterior approach to the disk. Such a procedure is typically employed in the cervical and lumbar spine. Alternatively, posterior lumbar interbody fusion involves the posterior insertion of an implant into the space between two lumbar vertebrae, following posterior excision of the disk.

A variety of implants and prostheses have been used to stabilize the spinal column. For example, the gap between adjacent vertebral bodies may be spanned with a rigid spacer that is comprised of bone graft material to facilitate growth of bone fusing the two vertebral bodies. Over time the patient's own bone grows into the graft, replacing and strengthening at least a portion of the original graft. A successful fusion stabilizes the spine, reduces pressure on the spinal cord and nerve roots, and reduces or eliminates back and peripheral pain.

Rigid bone graft spacers may be obtained from a variety of sources. An autograft may be harvested from the same individual for whom the implant is to be used. One common implant is referred to as a Cloward dowel, which is a circular graft made from the patient's illiac crest bone. The dowels are bicortical, having porous cancellous bone between two cortical surfaces. A cylindrical cutting tool is typically used to prepare the cervical site to receive the dowel, However, autologous implants are, in many situations, impractical and present risks to the patient because they require a second surgical site and potential damage to the bone from which the graft is harvested.

Alternatively, allografts may be obtained from other individuals (e.g., cadavers) from the same species. Xenografts may be obtained from other species. When derived from a human cadaver, grafts may be taken from long bones. Such grafts for use in spinal fusions include cortical rings derived from the femur, tibia, humerus, or fibula. Several tissue banks offer pre-shaped allograft cortical rings for this purpose. However, when using allografts, the potential of disease transmission and tissue rejection must be considered. Such grafts may undergo chemical treatment which sterilize the materials, remove potential antigenic proteins, and enhance their ability to promote bone in growth. A variety of graft materials, physical configurations, and chemical treatments are known in the art, including those disclosed in U.S. Pat. No. 6,277,149, Boyle et al., issued Aug. 21, 2001; and U.S. patent application Publication No. 2001/0039458, Boyer II, et al., published Nov. 8, 2001.

To be clinically useful, spinal bone grafts must be sterile, non-antigenic, easily fashioned from readily available sources, easily manipulated during surgical procedures, sufficiently strong to support and fix the spine immediately after surgery, and capable of promoting the growth of new bone after implantation. However, spinal implants among those known in the art lack one or more of these characteristics.

### SUMMARY OF THE INVENTION

The present invention provides spinal implants comprising substantially non-demineralized bone having at least one of several attributes, including a surface layer of demineralized bone, a beveled edge, and channels in the faces in contact with adjacent vertebral bodies. In one embodiment, the present invention provides a spinal implant comprising substantially non-demineralized bone and having a generally planar top surface, a generally planar bottom surface, and a side surface, wherein at least one of said top and said bottom surfaces is demineralized to a depth of from about 0.8 mm to about 3 mm. Preferably, the top and bottom surfaces of the implant are textured. Also preferably, the implant is disk shaped and comprises an insertion side extending at least about 10% of the circumference of said implant, wherein the edge formed by said insertion side and the top surface, and the edge formed by said insertion side and said bottom surface, are beveled. Also preferably at least one of said top and bottom surfaces comprises one or more radial channels.

It has been found that the implants of this invention afford benefits compared to implants among those known in the art. Such benefits include enhanced promotion of bone growth after implantation, increased structural stability after implantation, and improved handling and reduced breakage during surgical procedures. Specific benefits and embodiments of the present invention are apparent from the detailed description set forth herein. It should be understood, however, that the detailed description and specific examples, while indicating embodiments among those preferred, are intended for purposes of illustration only and are not intended to limit the scope of the invention.

### BRIEF DESCRIPTION OF THE DRAWINGS

FIG. 1 is a photograph exemplifying a implant embodiment of this invention.

FIG. 2 is a side view of an implant embodiment of this invention.

FIG. 3 is an orthogonal view of an implant embodiment of this invention.

FIG. 4 is a photograph exemplifying an implant embodiment of this invention, also depicting the attachment, in a preferred embodiment of use, of electrodes for electrical stimulation of bone growth.

FIGS. 5a and 5b are side views of implant embodiments of this invention.

FIG. 6 is a stereomicrograph of a cross section of an exemplary implant having a demineralized surface layer.

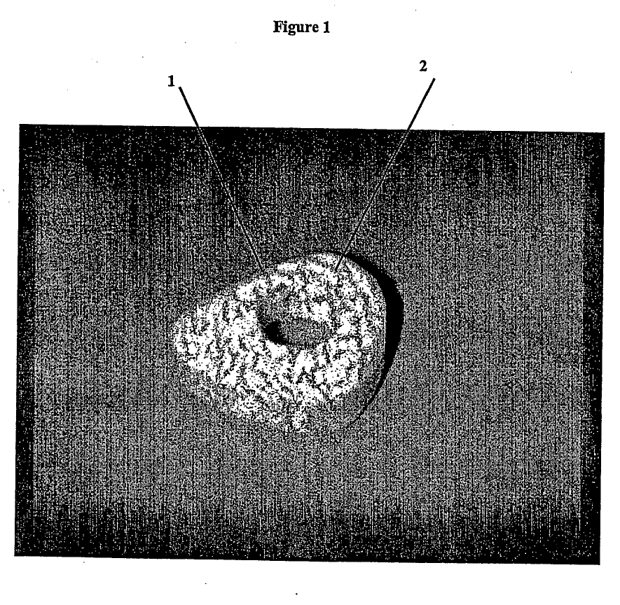

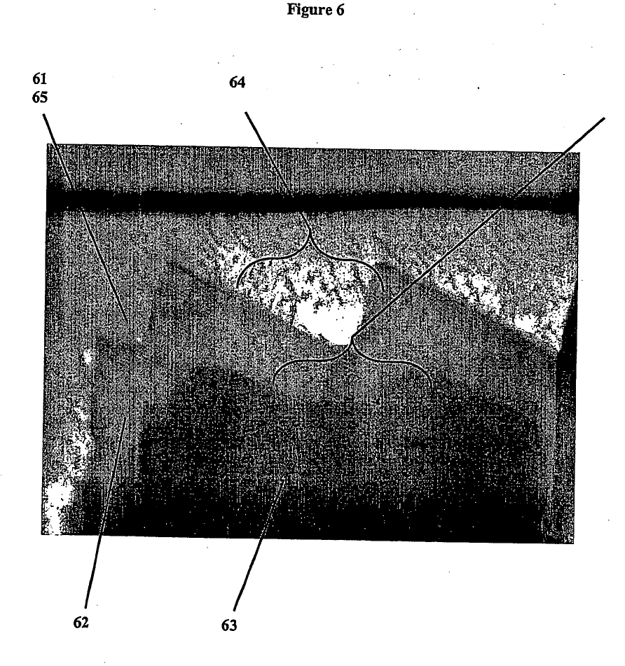

It should be noted that the pictures set forth herein, including those in FIGS. 1 and 6, are intended to exemplify the general characteristics of implants among those of this invention, for the purpose of the description of such embodiments herein. These pictures may not precisely reflect the characteristics of any given embodiment, and are not necessarily intended to define or limit specific embodiments within the scope of this invention.

### DETAILED DESCRIPTION OF THE INVENTION

The present invention encompasses certain novel spinal implants useful for the treatment of disorders in human or other animal subjects. In one embodiment, the implants of this invention are useful for implantation between two cervical vertebrae. In another embodiment, the implants are useful for implantation between two lumbar vertebrae. Specific materials to be used in the invention must, accordingly, be biocompatible. As used herein, such a “biocompatible” component is one that is suitable for use with humans and/or animals without undue adverse side effects (such as toxicity, irritation, and allergic response) commensurate with a reasonable benefit/risk ratio.

The implants of the present invention comprise non-demineralized bone. As referred to herein, “non-demineralized bone” comprises a material that is derived from the bones of human or other animal sources having a significant mineral content, preferably at levels equivalent to levels found in native bone. Such minerals in non-demineralized bone principally include hydroxyapatite. (As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention. Also as used herein, the word “include,” and its variants, is intended to be non-limiting, such that recitation of items in a list is not to the exclusion of other like items that may also be useful in the materials, compositions, devices, and methods of this invention.)

In one embodiment, non-demineralized bone is obtained from animal sources (i.e., for xenogenic implantation in a human subject) such as cows and pigs. In another preferred embodiment, non-demineralized bone is obtained from human cadavers (i.e., for allogenic implantation in a human subject). Such human bone material is available from a variety of tissue banks.

The implants may comprise cortical bone, cancellous bone, or a combination thereof. Cancellous bone is available in a range of porosities based on the location in the body from which the bone is harvested. Highly porous cancellous bone may be harvested from various areas such as the iliac crest, while less porous bone may be harvested from areas such as the tibial condyle femoral head, and calcaneus. Cortical bone may be obtained from long bones, such as the diaphyseal shaft of the femur and tibia. A preferred implant comprises cortical bone.

In another embodiment, an implant comprises a bone composite having two or more discrete layers or other regions of different bone materials, comprising cancellous, cortical bone or mixtures thereof. The regions in the composite may be joined in a variety of ways, including pins and chemical adhesion of the bone layers. Composites among those useful herein are disclosed in the following patent documents, all of which are incorporated by reference herein: U.S. Pat. No. 6,025,538; U.S. Pat. No. 6,200,347; U.S. Pat. No. 5,899,939; U.S. Pat. No. 6,123,731; U.S. Pat. No. 6,294,041; and U.S. Pat. No. 6,294,187.

The implants may have any of a variety of physical shapes and sizes, depending on the intended end use. Such implants include those that are substantially block shaped, wedge shaped (including a narrow ramp shape such as for use in posterior lumbar interbody fusions), cylindrically shaped, disk shaped and half-disk shaped. As referred to herein, an implant that is “substantially” of a particular shape (e.g., “substantially disk shaped”) has a gross morphology that is of the particular shape (e.g., circular) but may have variations in surface angles and textures. Such variations, in particular, may be as a result of variations in the source bone material. Thus, for example, an implant derived from a long bone is substantially disk shaped, although the circumference of the disk is irregular. A preferred implant embodiment is substantially disk shaped, more preferably ring shaped, having a cylindrical void substantially in the middle of the ring. Another preferred implant is half-ring shaped, made by cutting a ring essentially in half. Such rings and half-rings are preferably made from long bones, where the central cylindrical void in the ring is formed by the intramedullary canal of the bone. Such a ring shaped implant (1) is exemplified in FIG. 1.

Preferably the implant is from about 5 mm to about 40 mm wide and from about 5 to about 40 mm long (i.e., a diameter of from about 5 mm to about 40 mm for a disk shaped implant). In a preferred cervical implant embodiment, the width is from about 5 mm to about 14 mm, preferably from about 10 mm to about 14 mm. In a preferred lumbar implant embodiment, the width is from about 20 to about 40 mm. Preferably the implant has a height (i.e., in the direction substantially perpendicular to the top and bottom surfaces) of from about 5 mm to about 28 mm. In a preferred cervical implant embodiment, the height is from about 5 to about 15 mm. In a preferred lumbar implant embodiment, the height is from about 12 mm to about 28 mm.

In general, an implant comprises a generally planar top surface, a generally planar bottom surface, and a side surface. As referred to herein, a “generally planar” surface is substantially flat, having substantially two dimensional geometry considering the surface as a whole, although it may have surface irregularities in a third dimension. As referred to herein, the “plane defined by” the surface, is the plane in the two dimensions generally defined by the surface. As referred to herein, “top” and “bottom” are relative terms, indicating sides that are on opposite sides of the implant. In usage, for example, these surfaces are preferably in substantial contact with the vertebral bodies between which the implant is positioned. In one preferred embodiment the top and bottom surfaces are substantially parallel, i.e., the planes defined by the surfaces are non-intersecting. In another preferred embodiment, the plane defined by the top surface is angled from the plane defined by the bottom surface, to maintain a proper lordotic angle when implanted in a human or other animal subject. This is exemplified in FIG. 2, depicting a side view of an implant (21) having a top surface (22) and a bottom surface (23), wherein the plane (24) defining the top surface is angled from the plane (25) defining the bottom surface at an angle of approximately 6° (26). (For sake of convenience in FIG. 2, the angle (26) is depicted as being between top surface plane (24) and a plane (27) parallel to the bottom surface plane (25).) Preferably the angle is from about 1° to about 10°, more preferably from about 4° to about 8°, more preferably about 6°.

Preferably the top and bottom surfaces of the implant are textured. As referred to herein a “textured” surface has a rough or otherwise uneven surface. Such texturing may, for example, comprise grooves or other indentations into the surface of the implant, or bumps or other protrusions out of the surface of the implant. One embodiment of texturing comprises a knurled surface having an array of teeth. Grooves in the anterior-posterior, anterolateral, and lateral directions may be provided. Another embodiment comprises serrations in the surface, such as parallel sets of cross-cut (or perpendicular) serrations. Another embodiment comprises rows of teeth or grooves along the entire surface of the implant. Such teeth may be angled toward the anterior face of the graft, in a series of v-shaped parallel grooves having walls more than 90 degrees relative to the surface such that the peaks resulting from the grooves may or may not have flat tips. One example of surface texturing is exemplified in FIG. 1, wherein the surface of an implant (1) has a series of concentric grooves (2). Surface texturing useful herein is disclosed in the following patent documents, all of which are incorporated by reference herein: U.S. Pat. No. 6,143,033; U.S. Design Pat. No. D450121; U.S. Pat. No. 5,728,159; U.S. Pat. No. 5,989,289; U.S. Pat. No. 6,277,149; and PCT Patent Publication WO 99/09914.

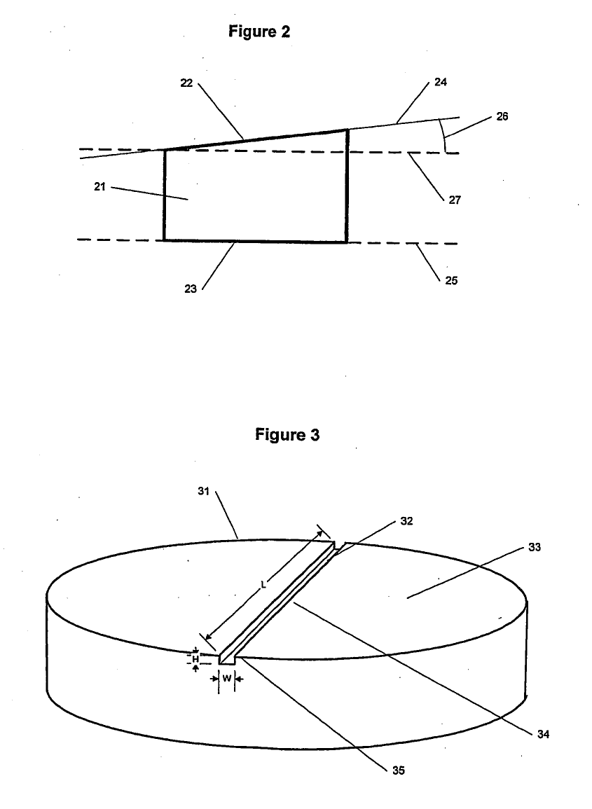

In a preferred embodiment, exemplified in FIG. 3, the implant (31) of the present invention comprises one or more radial channels (32) on one or both of the top (33) and bottom surfaces. As referred to herein, a “radial channel” is a groove or channel having a length (L), height (H), and width (W), which in its height dimension extends in a direction substantially perpendicular to the plane of the surface, and in its length dimension extends from a point substantially near the center (34) of the planar surface to a point substantially at an edge (35) of the surface. (The implant exemplified in FIG. 3 comprises two radial channels, which together extend across the entire diameter of the ring.) The profile of such channels (in a cross section perpendicular to the length dimension of the channel) may be, for example, substantially “V”-shaped, box shaped (with essentially perpendicular walls), or irregular. Preferably, a surface of the implant comprises from 1 to 10, more preferably from 4 to 8, radial channels.

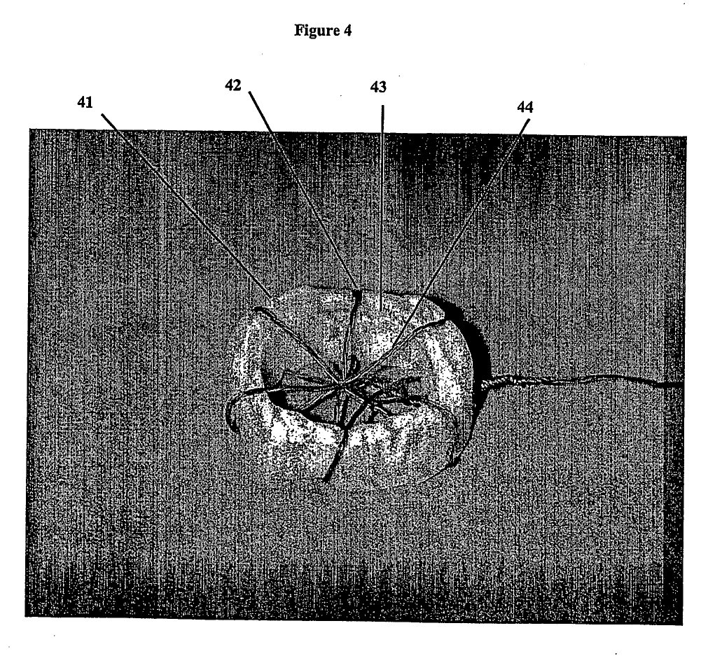

Preferably the channels are from about 0.2 mm to about 1 mm deep, and are from about 0.1 mm to about 18 mm wide. In one embodiment, the channels are preferably from 0.5 mm to about 15 mm wide. In another embodiment, the channels are preferably from about 0.2 mm to about 1 mm wide. In one embodiment, the grooves are configured to be suitable for use with an implantable electrical bone growth stimulator. Such devices are among those known in the art, and stimulate bone growth using direct current of preferably from about 10 μA to about 100 μA. See, e.g., A. Meril, “Direct Current Stimulation of Allograft in Anterior and Posterior Lumbar Interbody Fusions,” Spine 19:2393-2398 (1994), incorporated by reference herein. Electrodes for such devices may, for example, comprise wires or meshes. An implant embodiment of this invention having grooves suitable for use with a stimulator is exemplified in FIG. 4. In this example, the implant (41) has channels (e.g., 42) on both the top (43) and bottom (not shown) surfaces, and electrode wires (e.g., 44) are routed through the channels. A preferred stimulator is the SpF^{□} implantable Spinal Fusion Stimulator, marketed by EBI, L.P., Parsippany, N.J.

In another preferred embodiment, the implant comprises an insertion side, wherein the edge formed by said insertion side and the top surface, and the edge formed by said insertion side and said bottom surface, are beveled. As referred to herein, an “insertion side” is a side of the implant, preferably configured so as to face the direction of insertion between vertebral bodies during surgical implantation of the implant. For substantially block shaped implants, having four or more generally planar sides, the insertion side preferably comprises at least one side of the implant. For substantially disk shaped implants, preferably the insertion side comprises at least about 10%, more preferably at least about 35% of the circumference of the implant. In a preferred embodiment, the insertion side comprises all sides of a block shaped implant, or 100% of the circumference of a disk-shaped implant.

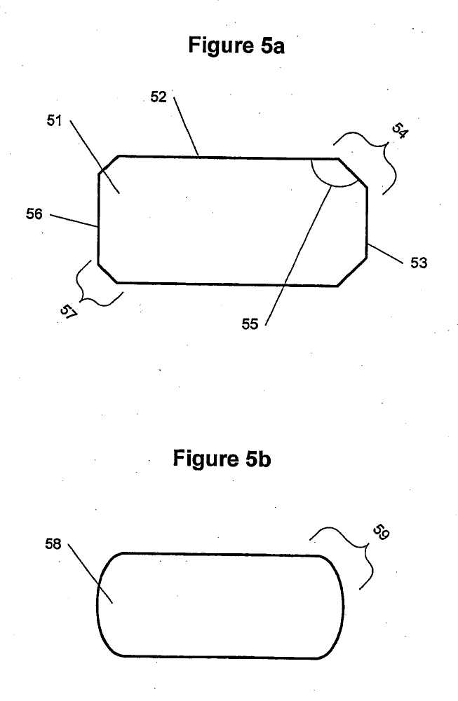

As referred to herein, a “beveled” edge refers to a rounded, flattened or other shaped edge substantially devoid of angles of intersection that are 90° or less. Such an embodiment of this invention is exemplified, in cross section, in FIG. 5a. The exemplified implant (51) has a top surface (52) and side insertion surface (53) forming an edge (54). The edge is beveled by flattening, such that the edge comprises the intersection of surfaces having an angle of intersection (55) greater than 90°. In this example, the implant comprises an insertion side and a non-insertion side (56), where the non-insertion side is also beveled (57), but the amount of non-insertion side (56) beveled is less than the amount of insertion side (53) that is beveled. In an alternate embodiment, exemplified in FIG. 5b, the edges of the implant (58) are beveled by rounding the edge (59).

In a preferred embodiment, at least one of the top and bottom surfaces of the implant comprises an osteoinductive surface layer having a depth of from about 0.8 mm to about 3 mm, preferably from about 1 mm to about 2 mm. As referred to herein, an “osteoinductive” surface layer is a layer of material which promotes the growth of bone material into the implant. A preferred osteoinductive layer comprises demineralized bone. Such embodiments of this invention preferably comprise:
(a) a body comprising substantially non-demineralized bone, having a generally planar top surface, a generally planar bottom surface, and a side surface; and
(b) a surface layer comprising demineralized bone;
   wherein said surface layer substantially covers one of said top and bottom surfaces, and wherein said surface layer is from about 0.8 mm to about 3 mm in depth. Preferably both the top and bottom surfaces are demineralized. Also preferably a side surface of the implant is demineralized, preferably all side surfaces of the implant are demineralized.

As referred to herein, “demineralized bone” is bone material from which a substantial portion of naturally-occurring minerals has been removed. Demineralized bone may be made in a variety of ways among those known in the art, preferably including subjecting a non-demineralized implant to a surface treatment that dissolves the minerals. Such implants comprise substantially non-demineralized bone and have a generally planar top surface, a generally planar bottom surface, and a side surface, wherein at least one of said top and said bottom surfaces is demineralized to a depth of from about 0.8 mm to about 3 mm.

A variety of chemical processing techniques may be used, including the use of acids, chelating agents and electrolysis. Preferred chemical treatments include those using hydrochloric acid, ethylene diamine tetraacetic acid (EDTA), or citric acid. The demineralization treatment removes the minerals contained in the natural bone, preferably leaving collagen fibers with bone growth factors including bone morphogenetic proteins (BMPs). Preferably the mineral content of the demineralized bone is from about 0% to about 5%, more preferably from about 0% to about 2%. (As referred to herein, all percentages are by weight unless otherwise specified.) Preferred demineralization techniques are described in K. U. Lewandrowski et al., “Kinetics of cortical bone demineralization: controlled demineralization—a new method for modifying cortical bone allografts,” J Biomed. Mater. Res., 31:365-372 (1996); K. U. Lewandrowski, et al., “An electron microscopic study on the process of acid demineralization of cortical bone,” Cal. Tiss. Int., 61:294-297 (1997); and K. U. Lewandrowski, et al., “Improved osteoinduction of cortical bone allografts: a study of the effects of laser perforation and partial demineralization,” J Orthop. Res., 15:748-756 (1997); all of which are incorporated by reference herein.

A preferred implant embodiment comprises:
(a) a body comprising substantially non-demineralized bone, having a generally planar top surface, a generally planar bottom surface, and a side surface; and
(b) a surface layer comprising demineralized bone;
   wherein the surface layer substantially covers one of said top and bottom surfaces, and wherein said surface layer is from about 0.8 mm to about 3 mm in depth, and wherein the surface layer is textured. Preferably the surface of the body which is covered by the textured surface layer is also textured. An example of such an implant is shown in FIG. 6. The exemplary implant (61) has a surface demineralized layer (62) and a non-demineralized core (63). The surface layer has texturing comprising grooves (e.g., 64). The core also has texturing comprising grooves (e.g., 65). Preferably, as depicted in this examples, the texturing of the core substantially corresponds to the texturing in the surface layer. In another embodiment, the texturing in the surface layer extends through the surface layer into the body, thereby forming texturing in the body. In a preferred embodiment, grooves in the demineralized surface layer extend through the surface layer into the body. In such an embodiment, at least a portion of the surface within the grooves of the body is exposed, i.e., is not covered with a layer of demineralized bone.

The present invention also provides methods for making a spinal implant, comprising:
(a) providing a implant body comprising non-demineralized bone having a generally planar top surface, a generally planar bottom surface, and a side surface; and
(b) demineralizing at least one of said top and bottom surfaces to a depth of from about 0.8 mm to about 3 mm. Preferably such methods additionally comprise the step of texturizing the implant body. In one embodiment, the texturizing step is performed prior to the demineralizing step. In another embodiment, the texturizing step is performed after the demineralizing step. In such an embodiment, preferably the texturing in the surface layer extends through the surface layer into the body, thereby forming texturing in the body. In such an embodiment where the texturing comprises grooves, at least a portion of the surface within the grooves of the body is exposed in the process of texturizing, such that the surface is not covered with a layer of demineralized bone.

The following are non-limiting Examples of the implants and methods of this invention.

### EXAMPLE 1

An implant is made by cutting an approximately 12 mm transverse segment from the fibula of a human cadaver to form an implant body. The body is substantially disk shaped, forming a ring having a diameter of approximately 12 mm. The top and bottom surfaces of the body are textured, to form concentric rings, using a concentric-arc ridge cutter. The edges of the body are then filed to form a bevel around the entire circumference of the segment.

The implant is then suspended in a vessel and immersed in 1.0 N HCl, at a ratio of 100 ml HCl per gram of bone. The acid is stirred, and maintained at ambient temperature (approximately 21° C.) for approximately two hours. The depth of demineralization is measured and determined to be approximately 1 mm. The implant is washed in buffered saline. The washing step is repeated three times, and the implant is then soaked in buffered saline for about 10 minutes. The implant is freeze dried and stored in a sterile container.

The implant is then surgically implanted between the cervical vertebrae of a human subject, after a diskectomy. X-rays of the subject show that, after 6 months, substantial bone growth has occurred into the implant, resulting in permanent fixation of the vertebrae.

### EXAMPLE 2

An implant is made by cutting an approximately 20 mm transverse segment from the femur of a human cadaver to form an implant body. The body is substantially disk-shaped, forming a ring having a diameter of approximately 25 mm. The top and bottom surfaces of the body are textures, to form concentric rings, using a concentric-arc ridge cutter. Six radial channels, approximately 0.5 mm wide and approximately 0.8 mm deep are cut into the top and bottom surfaces of the implant using a saw. The edges of the body are filed to form a bevel around the entire circumference of the segment.

The implant is then suspended in a vessel and immersed in 1.0 N HCl, at a ratio of 100 ml HCl per gram of bone. The acid is stirred, and maintained at ambient temperature (approximately 21° C.) for approximately seven hours. The depth of demineralization is measured and determined to be approximately 2 mm. The implant is washed in buffered saline. The washing step is repeated three times, and the implant is then soaked in buffered saline for about 10 minutes. The implant is then frozen and stored in a sterile container.

The implant is then wrapped with an electrode wire and surgically implanted between the lumbar vertebrae of a human subject, after a diskectomy. The electrode wire is connected to an electrical fusion stimulator, and the power source for the stimulator is implanted under the skin of the subject. X-rays of the subject show that, after 4 months, substantial bone growth has occurred into the implant, resulting in permanent fixation of the vertebrae.

### EXAMPLE 3

An implant is made by cutting an approximately 24 mm transverse segment from the femur of a human cadaver to form an implant body. The body is substantially disk shaped, forming a ring having a diameter of approximately 25 mm. The edges of the body are filed to form a bevel around the entire circumference of the segment. The body is then cut in half, to form two half-ring implants.

The implants are then suspended in a vessel and immersed in 1.0 N HCl, at a ratio of 100 ml HCl per gram of bone. The acid is stirred, and maintained at ambient temperature (approximately 21° C.) for approximately seven hours. The depth of demineralization is measured and determined to be approximately 2 mm. The implants are washed in buffered saline. The washing step is repeated three times, and the implants are then soaked in buffered saline for about 10 minutes.

The top and bottom surfaces of the body are textured, to form concentric rings, using a concentric-arc ridge cutter. The implants are freeze-dried and stored in a sterile container.

The implants are then surgically implanted between the lumbar vertebrae of a human subject, after diskectomy, in a posterior lumbar fusion. X-rays of the subject show that, after 6 months, substantial bone growth has occurred into the implants, resulting in permanent fixation of the vertebrae.

The examples and other embodiments described herein are exemplary and not intended to be limiting in describing the full scope of compositions and methods of this invention. Equivalent changes, modifications and variations of specific embodiments, materials, compositions and methods may be made within the scope of the present invention, with substantially similar results.

1. A spinal implant comprising substantially non-demineralized bone and having a generally planar top surface, a generally planar bottom surface, and a side surface, wherein at least one of said top and said bottom surfaces is demineralized to a depth of from about 0.8 mm to about 3 mm.

2. A spinal implant according to claim 1, wherein both of said top and bottom surfaces are demineralized.

3. A spinal implant according to claim 2, wherein said side surface is demineralized to a depth of from about 0.8 mm to about 3 mm.

4. A spinal implant according to claim 1, wherein at least one of said surfaces is demineralized to a depth of from about 1 mm to about 2 mm.

5. A spinal implant according to claim 2, wherein said top and bottom surfaces are textured.

6. A spinal implant according to claim I which is substantially disk shaped.

7. A spinal implant according to claim I which is substantially half-ring shaped.

8. A spinal implant according to claim 6, wherein said implant has a diameter of from about 5 mm to about 40 mm.

9. A spinal implant according to claim 8, wherein said implant is from about 5 mm to about 28 mm in height.

10. A spinal implant according to claim 9, wherein said top surface is substantially parallel to said bottom surface.

11. A spinal implant according to claim 9, wherein said top surface is angled from about 1 to about 10 degrees from the plane defined by said bottom surface.

12. A spinal implant according to claim 6, wherein at least one of said top and bottom surfaces comprises one or more radial channels.

13. A spinal implant according to claim 12, wherein both of said top and bottom surfaces comprise from 4 to 8 radial channels.

14. A spinal implant according to claim 12, wherein said channels are from about 0.2 mm to about 1 mm deep, and are from about 0.2 mm to about 15 mm wide.

15. A spinal implant according to claim 6, comprising an insertion side extending at least about 10% of the circumference of said implant, wherein the edge formed by said insertion side and the top surface, and the edge formed by said insertion side and said bottom surface, are beveled.

16. A spinal implant according to claim 15, wherein said insertion side extends at least about 35% of said circumference.

17. A spinal implant according to claim 15, wherein said insertion side extends about 100% of said circumference.

18. A spinal implant according to claim 1, for insertion between two cervical vertebrae in a human subject.

19. A spinal implant according to claim 1, for insertion between two lumbar vertebrae in a human subject.

20. A spinal implant according to claim 1, for implanting between two vertebrae of a human subject, comprising allogenic bone.

21. A spinal implant according to claim 1, for implanting between two vertebrae of a human subject, comprising xenogenic bone.

22. A spinal implant according to claim 1, comprising cortical bone.

23. A spinal implant according to claim 1, comprising cancellous bone.

24. A spinal implant according to claim 1, comprising a composite comprising cortical bone and cancellous bone.

25. A spinal implant, comprising
(a) a body comprising substantially non-demineralized bone, having a generally planar top surface, a generally planar bottom surface, and a side surface; and
(b) a surface layer comprising demineralized bone;
wherein said surface layer substantially covers one of said top and bottom surfaces, and wherein said surface layer is from about 0.8 mm to about 3 mm in depth.

26. A spinal implant according to claim 25, wherein said surface layer is textured with a plurality of grooves.

27. A spinal implant according to claim 25, wherein the surface of said body covered by said surface layer is textured with a plurality of grooves.

28. A spinal implant according to claim 27, wherein the grooves in said surface layer extend through said surface layer into said body thereby forming said grooves in said body.

29. A spinal implant comprising a substantially disk shaped body having a substantially planar top surface and a substantially planar bottom surface, wherein at least one of said top and bottom surfaces comprises one or more radial channels.

30. A spinal implant comprising a substantially ring shaped body having a substantially planar top surface, a substantially planar bottom surface, and an insertion side extending at least about 10% of the circumference of said implant, wherein the edge formed by said insertion side and the top surface, and the edge formed by said insertion side and said bottom surface, are beveled.

31. A method of making a spinal implant, comprising:
(a) providing a implant body comprising non-demineralized bone having a generally planar top surface, a generally planar bottom surface, and a side surface; and
(b) demineralizing at least one of said top and bottom surfaces to a depth of from about 0.8 mm to about 3 mm.

32. A method according to claim 29, additionally comprising the step of texturizing said implant body.

33. A method according to claim 32, wherein said texturizing step is performed prior to said demineralizing step.

34. A method according to claim 32, wherein said texturizing step is performed after said demineralizing step.

35. A method according to claim 31, additionally comprising freeze drying said implant after said demineralizing step.

36. A spinal implant made according to the method of claim 31.
